(19) United States
(12) Patent Application Publication
(10) Pub. No.: US 2018/0249914 A1
(43) Pub. Date: Sep. 6, 2018

(54) HUMAN HEALTH DETECTION SYSTEM

(71) Applicant: HUNG-TSUAN CHANG, TAINAN CITY (TW)

(72) Inventor: HUNG-TSUAN CHANG, TAINAN CITY (TW)

(21) Appl. No.: 15/448,770

(22) Filed: Mar. 3, 2017

(51) Int. Cl. A61B 5/0205; A61B 5/00

(52) U.S. Cl. CPC A61B 5/0205; A61B 5/053; A61B 5/0059; A61B 5/742

(57) ABSTRACT

A human health detection system detects the resistance value in each of twelve meridians of a human body to judge whether disorders occur in the corresponding organs or portions of the human body. Thus, a patient can understand his or her health condition. Furthermore, a doctor can use the detection result to more accurately diagnose problems in various portions of the human body, increasing the effect and efficiency of diagnosis.

### BACKGROUND OF THE INVENTION

The present invention relates to a medical auxiliary tool and, more particularly, to a human health detection system.

Bioelectrical impedance analysis in the research of medical impedance began in the late 19th century. Bioelectrical impedance analysis is a non-invasive detection technique for obtaining biological medical messages related to the physical and pathological conditions of the human body based on the electrical characteristics (impedance, dielectric constant, etc.) and their variance of biological tissues and organs. Generally, an electrode system is disposed on a skin surface of a person being examined, and a micro power is transmitted from a machine to the person to detect the corresponding impedance and its change. Then, related physical and pathological messages are obtained according to different applications. This technique is non-invasive, cheap, safe, non-toxic, harmless, and easy to operate, is easily accepted by medical workers and patients, and has wide applications. Bioelectrical impedance analysis has been widely used in our daily lives and uses the electrical impedance value to obtain the information of the biological body, such as body fat monitors, inspiration parameter measurement devices, skin humidity detectors, etc.

Since the impedances are different in different portions of the human body, the real conditions of the human body cannot be detected by a whole-body impedance detection method without considering the impedance differences in the upper limbs, torso, and lower limbs. Thus, in a more accurate measurement, the resistance value of each body portion can be measured to respond to the condition of each body portion. The resistance values of the upper limbs, torso, and lower limbs of different body sizes are also different. For example, the resistance of an upper limb can be up to about 250Ω, but the resistance of the torso is only about 25Ω. The upper limbs and the lower limbs of a healthy person are symmetric and have substantially the same resistance value. Nevertheless, a disabled person having defective limbs have different resistance values, and the difference could be large. Thus, when it is desired to obtain more accurate judgment result, measurements must be conducted in each portion rather than an overall resistance measurement.

According to the principles of traditional Chinese medicine and Chinese medicine health care, the meridians in the human body are the channels that connect the internal organs, coordinate of limb movements, convey qi and bloods, and adjust the function of each portion of the human body. Namely, the meridians and acupuncture points are the important passages that convey qi and bloods to each portion of the human body to maintain normal functions of the internal organs, the skin surface, and the skin, fresh, and bones of four limbs while providing necessary nutrition.

In 1949, Dr. Yoshio Nakatani of Kyoto University, Japan found certain skin resistance reaction patterns of patients suffering from various internal organ disorders based on the reaction points (which easily become conductive when in alignment) during research in skin resistance. Thus, the Applicant deems that, by combing the research of Dr. Yoshio Nakatani and the principles of traditional Chinese medicine and Chinese medicine health care, the resistance value in each meridian of the human body can be measured, and the change in the resistance value can be based to judge whether disorders occur in the organs or portions of the human body, such that a professional doctor can proceed subsequent diagnosis to more accurately understand and diagnose the health condition of the patient.

### BRIEF SUMMARY OF THE INVENTION

A human health detection system according to the present invention includes a resistance detector having an electrically conductive bar and an electrically conductive probe. The electrically conductive bar is adapted to be held by a person. The electrically conductive probe is adapted to abut one of twelve meridians of the person to form an electrical loop, thereby obtaining a meridian resistance value of the one of the twelve meridians. A processor is electrically connected to an output end and the resistance detector. A meridian resistance reference value database is electrically connected to the processor and includes a plurality of data items of resistance reference values of the twelve meridians. A resistance value comparison program is written to the processor and is executable by the processor to compare the meridian resistance value of the one of the twelve meridians with the plurality of data items of resistance reference values of a corresponding one of the twelve meridians. The comparison result is outputted via the output end.

In an example, the human health detection system further includes a blood pressure database and a blood pressure detector for detecting a blood pressure of the person to obtain a detected blood pressure value. The processor is electrically connected to the blood pressure detector. A blood pressure processing program is written to the processor. The blood pressure program is executable by the processor to store the detected blood pressure value into the blood pressure database and to output the blood pressure value via the output end, or to output each detected blood pressure value in the blood pressure database via the output end.

In an example, the human health detection system further includes a video unit. The video unit includes an image pickup device and a display. The image pickup device is adapted to pick up an image of the person to obtain image information. The processor is electrically connected to another video unit via a network. A video processing program is written to the processor and is executable by the processor to output the image information to the another video unit, to receive image data from the another video unit and to display the image data on the display.

In an example, the video unit further includes a microphone and a speaker. The microphone is adapted to pick up sounds of the person to obtain an audio signal. The video processing program is executable by the processor to transmit the audio signal to the another video unit, to receive another audio signal from the another video unit, and to output the another audio signal through the speaker.

In an example, each of the electrically conductive bar and the electrically conductive probe has a detection end made of copper.

In an example, one of the electrically conductive bar and the electrically conductive probe outputs a current of 200 (1±5%) μA.

Thus, by using the human health detection system according to the present invention, the resistance value of each meridian of the body of the user can be detected, and the meridian resistance value can be used to judge whether disorders occur in the corresponding organs or portions of the body of the user (e.g., by identifying whether the meridian resistance value exceeds a resistance value range). Furthermore, the detection result of the human health detection system can be used by medical workers to assist in accurate judgment of the physical health condition of patients, thereby improving the quality and efficiency of treatment.

The present invention will become clearer in light of the following detailed description of illustrative embodiments of this invention described in connection with the drawings.

### DESCRIPTION OF THE DRAWINGS

FIG. 1 is a diagrammatic view of a human health detection system according to the present invention.

FIG. 2 is a diagrammatic block diagram of the components of the human health detection system according to the present invention.

### DETAILED DESCRIPTION OF THE INVENTION

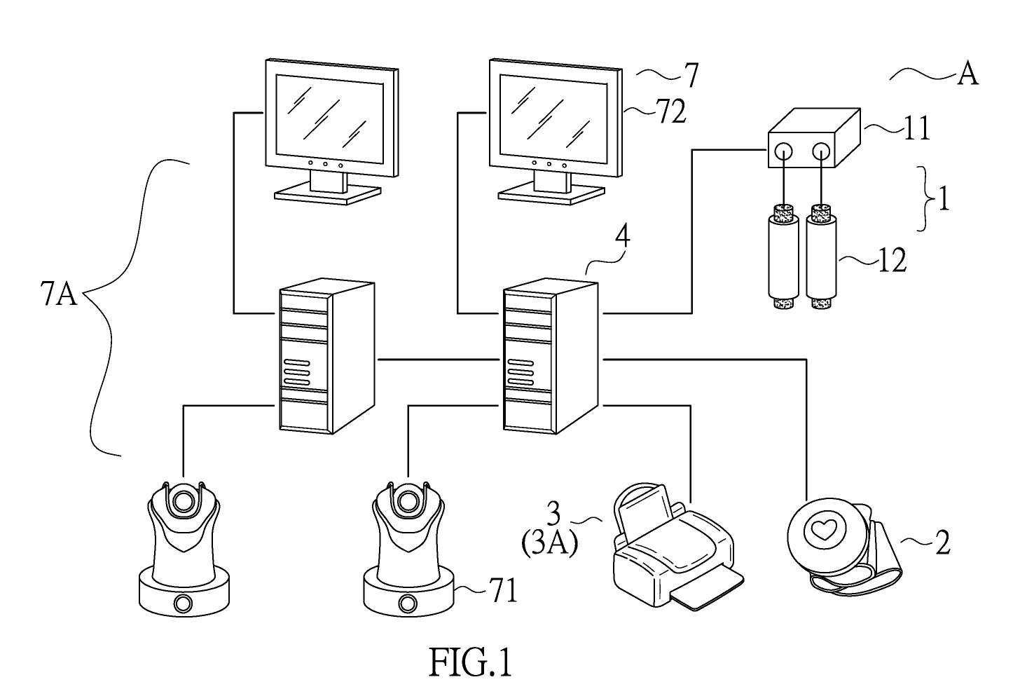

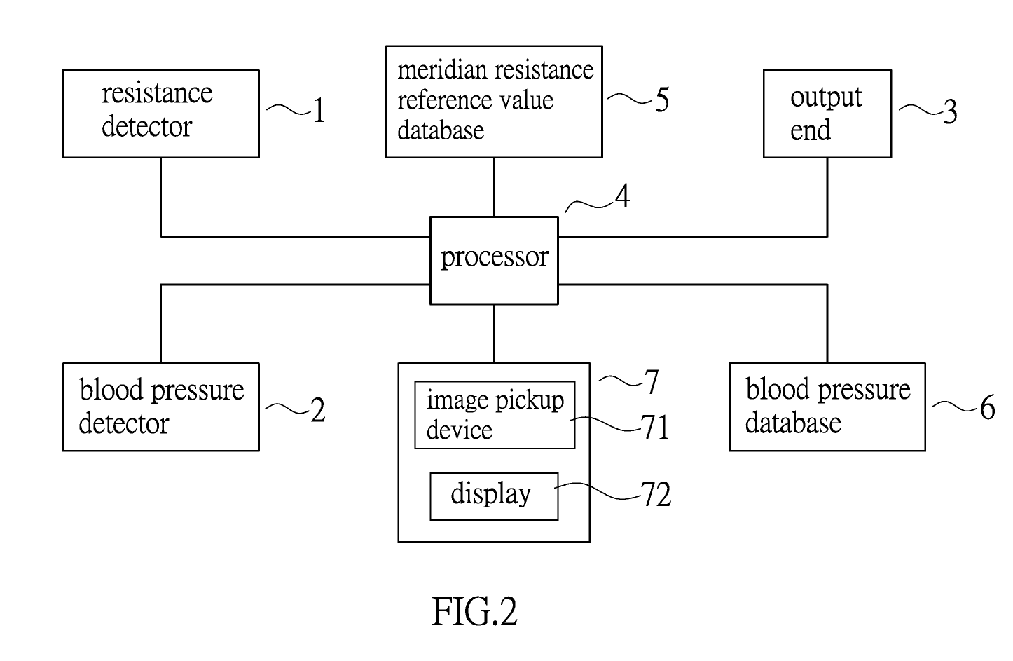

With reference to FIGS. 1 and 2, a human health detection system A according to the present invention includes a resistance detector 1, a blood pressure detector 2, an output end 3, and a processor 4. The resistance detector 1 includes an electrically conductive bar 11 and an electrically conductive probe 12. The electrically conductive bar 11 is adapted to be held by a person to be examined, such as a patient. The electrically conductive probe 12 is adapted to be held by a person, such as a doctor conducting the examination or a patient to be examined. A front end of the electrically conductive probe 12 is adapted to abut one of the twelve meridians of the person to be examined. One of the electrically conductive bar 11 and the electrically conductive probe 12 outputs a power to provide connection between the resistance detector 1 and a body of the person (forming an electrical loop), thereby detecting the resistance of the one of the twelve meridians to obtain a meridian resistance value. The electrically conductive bar 11 or the electrically conductive probe 12 outputs a current of 200 (1±5%) μA, which would not adversely affect the health of the elderly and children while providing a more accurate detection result. Each of the electrically conductive bar 11 and the electrically conductive probe 12 can have a detection end made of copper.

The twelve meridians referred to herein includes the lung meridian, the heart meridian, the heat protector meridian, the small intestine meridian, the lymph meridian, the large intestine meridian, the spleen meridian, the liver meridian, the kidney meridian, the urinary bladder meridian, the gall bladder meridian, and the stomach meridian.

With reference to FIGS. 1 and 2, the blood pressure detector 2 is used to detect a blood pressure of the person to be examined (such as a patient) to obtain a detected blood pressure value. The output end 3 outputs the detection result, such as the detected blood pressure value and the meridian resistance value. The output end 3 can be a display, a smart phone, a printer, etc. The person to be examined (such as a patient) or the person conducting the examination (such as a doctor) can know the detection result from the output end 3.

With reference to FIGS. 1 and 2, the processor 4 is electrically connected to the resistance detector 1, the blood pressure detector 2, the output end 3, a meridian resistance reference value database 5, and a blood pressure database 6. A resistance value comparison program and a blood pressure processing program are written to the processor 4. The resistance value comparison program is executable by the processor 4 to compare the meridian resistance value of the one of the twelve meridians detected by the resistance detector 1 with a plurality of data items of resistance reference values of a corresponding one of the twelve meridians in the meridian resistance reference value database 5, and the comparison result is outputted via the output end 3.

Thus, by using the human health detection system A according to the present invention, the resistance value of each meridian of the body of the user can be detected, and the meridian resistance value can be used to judge whether disorders occur in the corresponding organs or portions of the body of the user (e.g., by identifying whether the meridian resistance value exceeds a resistance value range). Furthermore, the detection result of the human health detection system A can be used by medical workers to assist in accurate judgment of the physical health condition of patients, thereby improving the quality and efficiency of treatment.

Furthermore, the corresponding organs of the above twelve meridians are: (1) lungs, nose, and skin; (2) heart sac and blood vessels; (3) heart, tongue, eyes, and armpits; (4) small intestines, eyes, and tongue; (5) lactiferous ducts, ears, and eyes; (6) large intestines, teeth, nose, and tongue; (7) spleen, stomach, and brain; (8) liver, eyes, and sex organ; (9) brain, eye, teeth, and nasal cavity; (10) urinary bladders, ears, nose, and eyes; (11) gall bladders, and head muscles; (12) stomach, milk lines, teeth, and mouth.

In the above comparison result, if the resistance value of a meridian exceeds a resistance value range, possible syndromes of the meridian could occur. For example, the related organs of the heart sac meridian include the heart sac and blood vessels. If the meridian resistance value of the heart protector meridian is higher than a preset resistance value range, swelling and painful eyes and congestion occur. If the meridian resistance value of the heart protector meridian is lower than the preset resistance value range, tachycardia and chest tightness occur. Thus, the user can clearly know the health conditions of each portion of the body by using the human health detection system A.

The blood pressure program is executable by the processor 4 to store the detected blood pressure value into the blood pressure database 6 and to output the detected blood pressure value via the output end 3. Alternatively, each detected blood pressure value in the blood pressure database 6 can be outputted via the output end 3. Thus, the user can record the blood pressure status every day, and the record can be used as a diagnosis reference for a doctor. Furthermore, a doctor using the human health detection system A can use the detection result of the meridians and cooperate the detected blood pressure value to more accurately judge the physical conditions of the patient, increasing the treatment effect.

For a patient suffering serious diseases and, thus, cannot easily move into and out of the consulting room, the human health detection system A according to the present invention can further use a network and/or a cloud system to proceed with video diagnosis with the doctor, providing easy diagnosis. Specifically, the human health detection system A further includes a video unit 7. The video unit 7 includes an image pickup device 71 and a display 72. The image pickup device 71 is adapted to pick up an image of the person to obtain image information. The processor 4 is directly or indirectly electrically connected to another video unit 7A via a network. A video processing program is written to the processor 4 and is executable by the processor 4 to output the image information to the another video unit 7A, to receive image data from the another video unit 7A, and to display the image data on the display 72. Through use of the video units 7 and 7A, the patient can see the diagnosis process of the doctor, which not only relieves the patient but creates mutual trust between the patient and the doctor.

To provide easy diagnosis for the doctor or to guide the image pick-up position of the image pickup device 71, the video unit 7 can further include a microphone and a speaker. The microphone is adapted to pick up sounds of the person to obtain an audio signal. The video processing program is executable by the processor 4 to transmit the audio signal to the another video unit 7A, to receive another audio signal from the another video unit 7A, and to output the another audio signal through the speaker. Furthermore, the number of the another video unit 7A connected by the network to the processor 4 can be more than one, such that a plurality of doctors can proceed with diagnosis on the patient via the network and/or the cloud system.

Although specific embodiments have been illustrated and described, numerous modifications and variations are still possible without departing from the scope of the invention. The scope of the invention is limited by the accompanying claims.

What is claimed is:

1. A human health detection system comprising:
a resistance detector including an electrically conductive bar and an electrically conductive probe, with the electrically conductive bar adapted to be held by a person, with the electrically conductive probe adapted to abut one of twelve meridians of the person to form an electrical loop, thereby obtaining a meridian resistance value of the one of the twelve meridians;
an output end; and
a processor electrically connected to the output end and the resistance detector, with a meridian resistance reference value database electrically connected to the processor and including a plurality of data items of resistance reference values of the twelve meridians, wherein a resistance value comparison program is written to the processor and is executable by the processor to compare the meridian resistance value of the one of the twelve meridians with the plurality of data items of resistance reference values of a corresponding one of the twelve meridians, and a comparison result is outputted via the output end.

2. The human health detection system as claimed in claim 1, further comprising a blood pressure database and a blood pressure detector for detecting a blood pressure of the person to obtain a detected blood pressure value, with the processor electrically connected to the blood pressure detector, with a blood pressure processing program written to the processor, with the blood pressure program executable by the processor to store the detected blood pressure value into the blood pressure database and to output the blood pressure value via the output end, or to output each detected blood pressure value in the blood pressure database via the output end.

3. The human health detection system as claimed in claim 1, further comprising a video unit, with the video unit including an image pickup device and a display, with the image pickup device adapted to pick up an image of the person to obtain image information, with the processor electrically connected to another video unit via a network, with a video processing program written to the processor and executable by the processor to output the image information to the another video unit, to receive image data from the another video unit and to display the image data on the display.

4. The human health detection system as claimed in claim 3, with the video unit further including a microphone and a speaker, with the microphone adapted to pick up sounds of the person to obtain an audio signal, with the video processing program executable by the processor to transmit the audio signal to the another video unit, to receive another audio signal from the another video unit, and to output the another audio signal through the speaker.

5. The human health detection system as claimed in claim 1, wherein each of the electrically conductive bar and the electrically conductive probe has a detection end made of copper.

6. The human health detection system as claimed in claim 5, wherein one of the electrically conductive bar and the electrically conductive probe outputs a current of 200 (1±5%) μA.
